(19) United States
(12) Patent Application Publication
(10) Pub. No.: US 2021/0393192 A1
(43) Pub. Date: Dec. 23, 2021

(54) MUSCLE FATIGUE EVALUATION METHOD AND MUSCLE FATIGUE EVALUATION SYSTEM

(71) Applicant: TORAY ENGINEERING CO., LTD., Tokyo (JP)

(72) Inventors: Yusuke SAKAUE, Otsu-shi (JP); Chisa INAKA, Otsu-shi (JP); Kazushige GOTO, Kusatsu-shi (JP)

(21) Appl. No.: 17/462,468

(22) Filed: Aug. 31, 2021

Related U.S. Application Data
(63) Continuation of application No. PCT/JP2020/010076, filed on Mar. 9, 2020.

(30) Foreign Application Priority Data
Mar. 19, 2019 (JP) 2019-051809
Aug. 21, 2019 (JP) 2019-151181

(51) Int. Cl. A61B 5/00; A61B 5/053; A61B 5/296

(52) U.S. Cl. CPC A61B 5/4519; A61B 5/296; A61B 5/053

(57) ABSTRACT

At least two electrodes are disposed at a predetermined interval on a living body surface, a first voltage V₁ generated when a first external resistor is connected in parallel between the two electrodes, and a second voltage V₂ generated when a second external resistor is connected in parallel between the two electrode are measured, a bioimpedance between the two electrodes at a muscle site under the living body surface is calculated based on a voltage ratio V₁/V₂ between the first voltage V₁ and the second voltage V₂, and local muscle fatigue at the muscle site is evaluated based on a change over time in the calculated bioimpedance.

### CROSS-REFERENCE TO RELATED APPLICATIONS

This application is a continuation application of PCT International Application No. PCT/JP2020/010076 filed on Mar. 9, 2020, which claims priority to Japanese Patent Application No. 2019-051809 filed on Mar. 19, 2019 and 2019-151181 filed on Aug. 21, 2019. The entire disclosures of PCT International Application No. PCT/JP2020/010076 and Japanese Patent Application Nos. 2019-051809 and 2019-151181 are hereby incorporated herein by reference.

### BACKGROUND

### Field of the Invention

The present invention relates to a muscle fatigue evaluation method and a muscle fatigue evaluation system for evaluating fatigue of a specific muscle by measuring the impedance in a living body.

### Background Information

As a method for easily evaluating a muscle fatigue state, a method to measure the impedance in a living body by passing a high-frequency current from the outside into the living body is known (see Japanese Patent Application Publication No. 2004-49789 (Patent Literature 1), for example).

However, this method requires a high-frequency current to flow from the outside into the living body, which poses the risk of an electric shock or the like. In addition, it is difficult to evaluate the fatigue of a specific muscle because the overall impedance in the living body is measured.

By contrast, a method to measure an electromyogram is known as a method for non-invasively evaluating a muscle fatigue state (see Tomohiro Kizuka, Tadashi Masuda, Tohru Kiryu, and Tsugutake Sadoyama, “Practical Usage of Surface Electromyogram,” Tokyo Denki University Press, March 2006, pp. 60-62 (Non-Patent Literature 1), for example). This method allows the fatigue of a specific muscle to be evaluated by measuring changes in the amplitude and the center frequency of the electromyogram.

### SUMMARY

However, since an electromyogram is a record of biological signals obtained by causing a muscle to contract, the amplitude and the center frequency change when the magnitude of the load applied to the muscle is changed. Therefore, muscle fatigue can be evaluated only when a load of a certain magnitude has been applied to the muscle. That is, an electromyogram, by itself, is not an index for evaluating muscle fatigue that occurs continuously in a state in which a non-constant load is applied to the muscle.

The present invention is conceived in the light of this point, and a main object thereof is to provide a non-invasive muscle fatigue evaluation method and muscle fatigue evaluation system with which the muscle fatigue that occurs when a non-constant load is applied to a specific muscle can be evaluated.

A muscle fatigue evaluation method according to the present invention comprises disposing at least two electrodes at a predetermined interval on a living body surface, measuring a first voltage V₁ generated when a first external resistor is connected in parallel between the two electrodes and a second voltage V₂ generated when a second external resistor is connected in parallel between the two electrodes, calculating a bioimpedance between the two electrodes at a muscle site under the living body surface based on a voltage ratio V₁/V₂ between the first voltage V₁ and the second voltage V₂, and evaluating local muscle fatigue at the muscle site based on a change over time in the calculated bioimpedance.

A muscle fatigue evaluation system according to the present invention comprises at least two electrodes disposed at a predetermined interval on a living body surface, a connection circuit configured to switchably connect a first external resistor and a second external resistor in parallel between the two electrodes, a voltage measurement circuit configured to measure a first voltage V₁ generated when the first external resistor is connected in parallel between the two electrodes by the connecting circuit, and a second voltage V₂ generated when the second external resistor is connected in parallel between the two electrodes by the connecting circuit, and an impedance calculator configured to calculate a bioimpedance between the two electrodes at a muscle site under the living body surface based on a voltage ratio V₁/V₂ between the first voltage V₁ and the second voltage V₂, local muscle fatigue at the muscle site being evaluated based on a change over time in the calculated bioimpedance.

The present invention allows muscle fatigue that occurs when a non-constant load is applied to a specific muscle to be continuously and non-invasively evaluated.

### BRIEF DESCRIPTION OF THE DRAWINGS

Referring now to the attached drawings which form a part of this original disclosure:

FIG. 1 is a diagram schematically showing a muscle fatigue evaluation method according to an embodiment of the present disclosure;

FIG. 2 is an equivalent circuit diagram in the state shown in FIG. 1;

FIGS. 3A, 3B and 3C are graphs of the results of examining the relation between muscle fatigue and bioimpedance;

FIGS. 4A and 4B are graphs schematically showing the relation between the change in the amount of water in a muscle and the change in bioimpedance;

FIG. 5 is a graph of the results of examining acute muscle fatigue and chronic muscle fatigue;

FIG. 6 is a diagram showing an arrangement of the two electrodes; and

FIG. 7 is a block diagram showing a configuration of a muscle fatigue evaluation device.

### DETAILED DESCRIPTION OF EMBODIMENTS

Embodiments of the present invention will now be described in detail with reference to the drawings. The present invention is not limited to the following embodiments. Also, suitable changes are possible to the extent that such changes do not deviate from the range in which the effect of the present invention is exhibited.

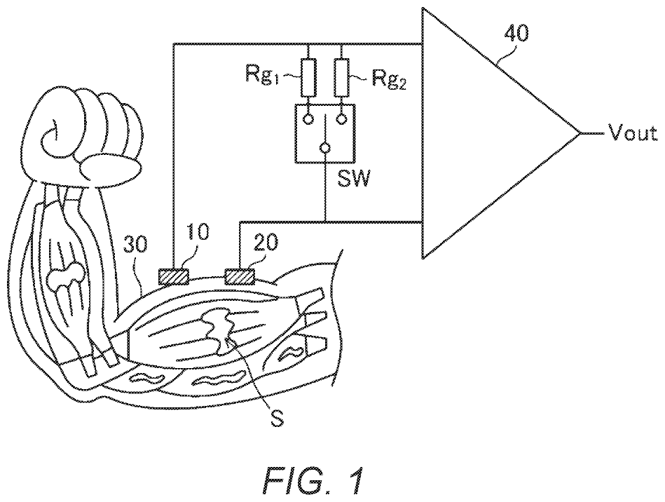

FIG. 1 is a diagram schematically showing a muscle fatigue evaluation method according to an embodiment of the present disclosure.

As shown in FIG. 1, two electrodes 10 and 20 are disposed at a predetermined interval on a living body surface 30. Here, an example is given in which the two electrodes 10 and 20 are attached to the surface of an upper arm. The voltage generated between the electrodes 10 and 20 is measured by being amplified by an amplifier (voltage measurement means or voltage measurement circuit) 40. Also, a first external resistor Rg1 and a second external resistor Rg2 are disposed in parallel between the two electrodes 10 and 20. A switch (connecting means or connecting circuit) SW switches between a state in which the first external resistor Rg1 is connected in parallel between the electrodes 10 and 20 and a state in which the second external resistor Rg2 is connected in parallel between the electrodes 10 and 20.

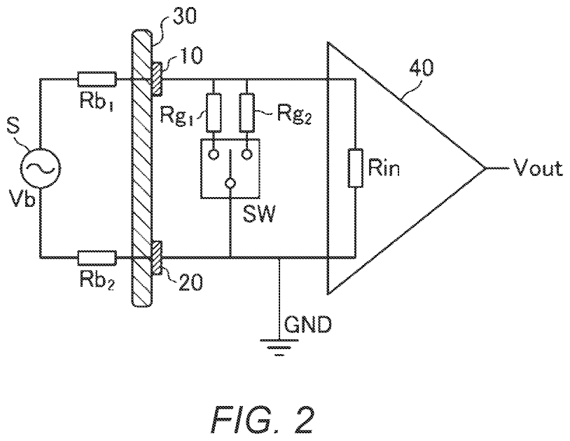

FIG. 2 shows an equivalent circuit diagram of the state shown in FIG. 1.

Here, Vb is the myoelectric potential at the muscle site (brachial muscle) under the living body surface 30 between the two electrodes 10 and 20. This myoelectric potential Vb is generated when the upper arm 30 is exercised, that is, when a load is applied to the brachial muscle.

Also, Rb1 and Rb2 respectively indicate the bioimpedance between a signal source S that generates the myoelectric potential Vb, and the electrodes 10 and 20. The bioimpedance will be described in detail in the description of the relation to muscle fatigue given below. Rin indicates the input resistance of the amplifier 40. The voltage generated between the two electrodes 10 and 20 is amplified by the amplifier 40 and measured as the output voltage Vout.

In the equivalent circuit diagram shown in FIG. 2, the first voltage V₁ generated when the first external resistor Rg1 is connected in parallel between the two electrodes 10 and 20 is given by the formula (2).

$${V}_{1} = \frac{{R}_{g 1}}{{R}_{b 1} + {R}_{b 2} + {R}_{g 1}} {V}_{b} \qquad ( 2 )$$

The second voltage V₂ generated when the second external resistor Rg2 is connected in parallel between the two electrodes 10 and 20 is given by the formula (3).

$${V}_{2} = \frac{{R}_{g 2}}{{R}_{b 1} + {R}_{b 2} + {R}_{g 2}} {V}_{b} \qquad ( 3 )$$

Therefore, using the formulas (2) and (3), the bioimpedance Zb (=Rb1+Rb2) between the two electrodes 10 and 20 at the muscle site (brachial muscle) under the living body surface 30 is obtained by the following formula (1).

$${Z}_{b} = {R}_{b 1} + {R}_{b 2} = \frac{{R}_{g 1} \cdot {R}_{g 2}}{{R}_{g 1} - {R}_{g 2} \cdot \frac{{V}_{1}}{{V}_{2}}} ( \frac{{V}_{1}}{{V}_{2}} - 1 ) \qquad ( 1 )$$

That is, using the formula (1), the bioimpedance Zb between the two electrodes 10 and 20 at the muscle site (brachial muscle) under the living body surface 30 can be calculated based on the voltage ratio (V₁/V₂) between the first voltage V₁ and the second voltage V₂.

On the other hand, it is well known that the blood lactate concentration increases when a load is applied to muscles to the point that muscle fatigue occurs, but it is also known that the water content in muscles also increases. Therefore, when the bioimpedance at the fatigued muscle site is measured, it can be predicted that the bioimpedance will be lower than normal.

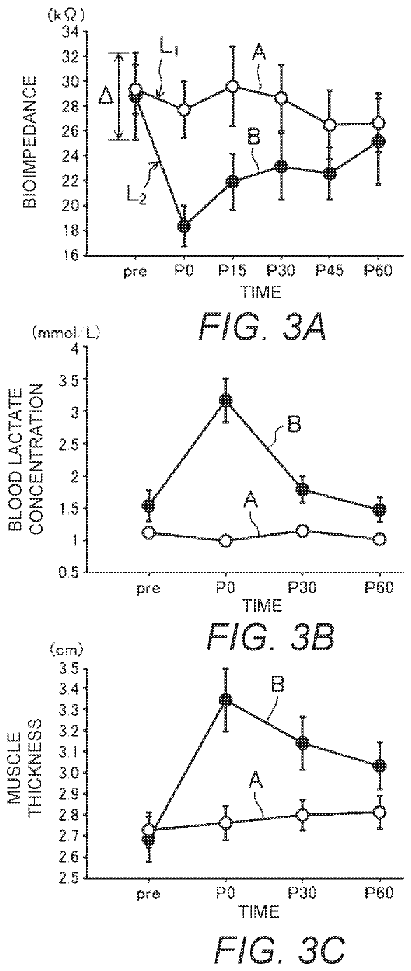

FIGS. 3A, 3B and 3C are graphs of the results of examining the relation between muscle fatigue and bioimpedance. In the test, nine healthy adult men were used as subjects, each of whom used a dumbbell with a weight of 60% of the subject's maximum muscle strength to perform 12 arm curls, and this was repeated for five sets to fatigue the brachial muscle.

FIG. 3A is a graph showing the results of measuring the bioimpedance of a subject who performed the above exercise, using the muscle fatigue evaluation method shown in FIGS. 1 and 2. The bioimpedance was measured by attaching two electrodes at an interval of 3.5 cm along the muscle fibers of the brachial muscle in the muscle fatigue evaluation method shown in FIGS. 1 and 2.

Here, the resistance value of the first external resistor Rg1 was set so that the voltage ratio V₁/V₂ between the first voltage V₁ and the second voltage V₂ was about 0.3. The resistance value of the second external resistor Rg2 was set to infinity. Then, the first voltage V₁ and the second voltage V₂ were measured while using a 4.0-kg dumbbell and holding the elbow joint angle at 90°.

When the resistance value of the second external resistance Rg2 is set to infinity, the above formula (3) is V₂≈Vb, so the bioimpedance Zb can be obtained by the following formula (4).

$${Z}_{b} = \frac{{V}_{2} - {V}_{1}}{{V}_{1}} {R}_{g 1} \qquad ( 4 )$$

In FIG. 3A, the horizontal axis indicates time (minutes) and the vertical axis indicates bioimpedance (kΩ). Graph A in the drawing shows the bioimpedance measured when the above exercise was not performed, and graph B shows the bioimpedance measured when the above exercise was performed. The bioimpedance in the graphs shows the average for nine people.

The bioimpedance at P0 on the horizontal axis indicates the value immediately after the exercise, and the bioimpedance at P15 to P60 indicates the values measured every 15 minutes after the exercise ended.

As shown in FIG. 3A, the bioimpedance can be seen to decrease greatly immediately after the above exercise (P0). It can also be seen that the bioimpedance gradually returns as time passes (P15 to P60) after the exercise is finished.

FIG. 3B is a graph showing the results of measuring the blood lactate concentration by taking blood samples from the subjects at P0, P30, and P60. Graph A in the drawing shows the blood lactate concentration measured when the above exercise was not performed, and graph B shows the blood lactate concentration measured when the exercise was performed. The blood lactate concentration shown in the drawings is the average for nine people.

As shown in FIG. 3B, it can be seen that the blood lactate concentration is greatly increased immediately after the above exercise (P0). It can also be seen that the blood lactate concentration gradually returns as time passes (P30, P60) after the exercise is finished.

FIG. 3C is a graph of the results of measuring the muscle thickness in the upper arm of the subject at P0, P30, and P60. Graph A in the drawing shows the muscle thickness measured when the above exercise was not performed, and graph B shows the muscle thickness measured when exercise was performed. The muscle thickness in the drawings shows the average value for nine people.

As shown in FIG. 3C, it can be seen that the muscle thickness is greatly increased immediately after the above exercise (P0). It can also be seen that the muscle thickness gradually returns as time passes (P30, P60) after the exercise is finished.

It can be seen from the above results that the change in bioimpedance when a non-constant load is applied to the muscle, such as in biceps curls, is strongly correlated with the change in blood lactate concentration and the change in muscle thickness. That is, the fatigue of a specific muscle can be ascertained as the change in the amount of water in the muscle, and this tells us that the change in bioimpedance can serve as an index reflecting muscle fatigue.

As explained above, when a load to is applied to a muscle and muscle fatigue occurs, the amount of water in the muscle changes, and the fatigue in a specific muscle can be evaluated in real time by ascertaining the change in the amount of water in the muscle as the change in bioimpedance.

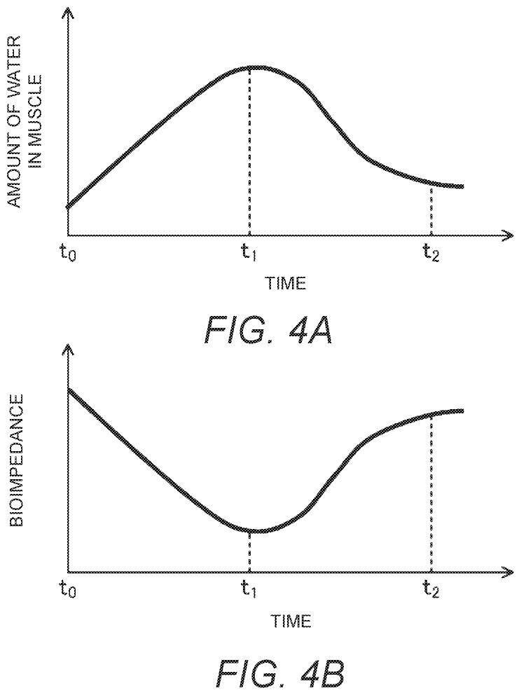

FIGS. 4A and 4B are graphs schematically showing the relation between the change in the amount of water in a muscle (FIG. 4A) and the change in the bioimpedance (FIG. 4B). As shown in FIGS. 4A and 4B, when a load to is applied to the muscle and muscle fatigue occurs, the amount of water in the muscle increases (time t₀ to t₁), and the bioimpedance decreases along with this (time t₀ to t₁). When the load on the muscle is stopped, the amount of water in the muscle returns to its original state (time t₁ to t₂), and along with this, the bioimpedance also returns to its original state (time t₁ to t₂). That is, measuring the change over time in the bioimpedance reveals that muscle fatigue accumulates from the time t₀ to t₁, and is restored between the times t₁ and t₂. Consequently, it possible to evaluate in real time the muscle fatigue that occurs when a load is applied to muscles.

As shown in graph A in FIG. 3A, the bioimpedance may change over time due to factors other than muscle fatigue, even when no exercise is performed. Therefore, when muscle fatigue is evaluated based on the change over time in bioimpedance, it is necessary to distinguish from the change over time due to factors other than muscle fatigue.

Usually, as shown in FIG. 3A, the amount of change over time (slope) L₁ in bioimpedance when exercise is not performed is less than the amount of change over time (slope) L₂ in bioimpedance when exercise is performed. Typically, when the bioimpedance immediately after exercise changes by 20% or more with respect to the bioimpedance before exercise, this is believed to be due to muscle fatigue.

On the other hand, when the change over time in the bioimpedance is 20% or less, this is not believed to be due to muscle fatigue. Therefore, muscle fatigue can be correctly evaluated by determining that muscle fatigue has occurred when an amount of the change over time in the bioimpedance calculated after exercise is equal to or greater than a predetermined value.

Also, as shown in FIG. 3A, a certain fluctuation Δ occurs in the calculation of the bioimpedance. Accordingly, when muscle fatigue is evaluated based on the change over time in the calculated bioimpedance, it is necessary to distinguish from the change over time due to fluctuation in the bioimpedance. Therefore, the bioimpedance is calculated a number of times before exercise to obtain the fluctuation of the calculated bioimpedance, and muscle fatigue is evaluated based on the change over time when the bioimpedance calculated after exercise is greater than or equal to the fluctuation, which allows muscle fatigue to be correctly evaluated.

On the other hand, it is known that when a load is applied to a muscle, transient muscle fatigue (acute muscle fatigue) initially occurs, after which the muscle fatigue recovers, and then muscle fatigue (chronic muscle fatigue) occurs again.

The muscle fatigue evaluation method of this embodiment allows these acute muscle fatigue and chronic muscle fatigue to be evaluated in real time.

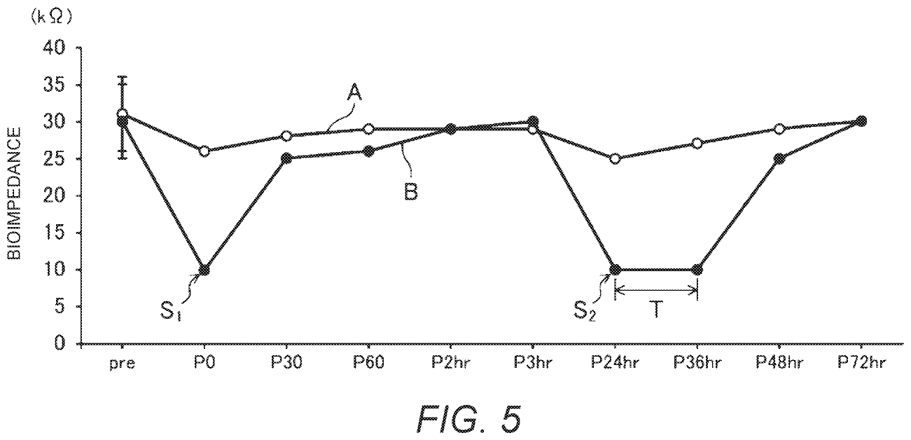

FIG. 5 is a graph of the results of measuring the bioimpedance of a subject who has exercised, using the muscle fatigue evaluation method shown in FIGS. 1 and 2. The bioimpedance was measured by the same method as that shown in FIG. 3A.

In FIG. 5, the horizontal axis indicates time and the vertical axis indicates bioimpedance (kΩ). Graph A in the drawing shows the bioimpedance measured when exercise was not performed, and graph B shows the bioimpedance measured when exercise was performed.

The bioimpedance at pre on the horizontal axis indicates the value before exercise, and the bioimpedance at P0 on the horizontal axis indicates the value immediately after exercise is finished. P30, P60, P2hr, P3hr, P24hr, P36hr, P48hr, and P72hr on the horizontal axis indicate 30 minutes, 60 minutes, 2 hours, 3 hours, 24 hours, 36 hours, 48 hours, and 72 hours after exercise, respectively.

As shown in FIG. 5, immediately after exercise is finished (P0), the bioimpedance can be seen to have greatly decreased, reaching the minimal value S₁. After this, it can be seen that the bioimpedance gradually returns as time passes (P30 to P60), reaching the pre-exercise value about 2 hours later (P2hr).

As more time passes, the bioimpedance again greatly decreases after about 3 hours (P3hr), and can be seen to have reached the second minimal value S₂ after about 24 hours (P24hr). The second minimal value S₂ continues for about 12 hours (T), and after 36 hours (P36hr), the bioimpedance can be seen to have gradually returned to the pre-exercise value after about 72 hours (P72hr).

In the change over time in the bioimpedance shown in FIG. 5, the muscle fatigue at the point when the initial minimal value S₁ is reached can be determined to be acute muscle fatigue. Also, the muscle fatigue at the point when the next minimal value S₂ is reached after the initial minimal value S₁ can be determined to be chronic muscle fatigue.

Also, in the change over time in the bioimpedance shown in FIG. 5, the duration of the minimal value S₁ is the time it takes from the rapid decrease to the rapid increase, whereas the duration of the minimal value S₂ is the time it takes from the rapid decrease to the rapid increase after a certain amount of time has elapsed. That is, the duration of the minimal value S₂ is longer than the duration of the minimal value S₁. Therefore, muscle fatigue when the duration of the minimal value S₁ is short can be determined to be acute muscle fatigue, and muscle fatigue when the duration of the minimal value S₂ is long can be determined to be chronic muscle fatigue.

Thus, with the muscle fatigue evaluation method in this embodiment, acute muscle fatigue and chronic muscle fatigue that occur after applying a load to a muscle can be evaluated in real time and non-invasively. In particular, evaluating chronic muscle fatigue used to demand a high degree of specialized knowledge, but in this embodiment, chronic muscle fatigue can be evaluated by a simple method. As a result, it is possible to prevent the risk of injury or the like posed by training in a state in which chronic muscle fatigue remains, and to prevent a decrease in the training effect, as well as the occurrence of overtraining due to excessive training.

The muscle fatigue evaluation method in this embodiment comprises disposing at least two electrodes 10 and 20 at a predetermined interval on the living body surface, measuring the first voltage V₁ generated when the first external resistor Rg1 is connected in parallel between the two electrodes 10 and 20 and the second voltage V₂ generated when the second external resistor Rg2 is connected in parallel between the two electrodes 10 and 20, calculating the bioimpedance Zb between the two electrodes 10 and 20 at a muscle site under the living body surface based on the voltage ratio V₁/V₂ between the first voltage V₁ and the second voltage V₂, and evaluating the local muscle fatigue at the muscle site based on the change over time in the calculated bioimpedance Zb.

This allows muscle fatigue that occurs when a non-constant load is applied to a specific muscle to be evaluated in real time. Also, since muscle fatigue at a specific muscle site to be evaluated for fatigue can be evaluated merely by disposing two electrodes at this site, muscle fatigue can be evaluated accurately and non-invasively.

Because the present disclosure provides this effect, muscle fatigue can be evaluated for each specific muscle in daily training, for example, so more effective training can be performed. Also, since muscle fatigue can be evaluated in real time, this prevents the exacerbation of a condition or the occurrence of injuries caused by overtraining.

The present disclosure was described above in terms of a preferred embodiment, but this description is not a limitation, and various modifications are, of course, possible.

For example, in the above embodiment, the two electrodes 10 and 20 are disposed on the living body surface, but a ground electrode may be disposed on the living body surface and the voltage generated between the two electrodes 10 and 20 may be measured with a differential amplifier 40. Here again, the bioimpedance between the two electrodes 10 and 20 can be obtained from the above formula (1). Also, since the first voltage V₁ and the second voltage V₂ are measured by being amplified by the differential amplifier 40 after taking the difference therebetween, external noise can be removed. This allows the bioimpedance Zb to be measured more accurately.

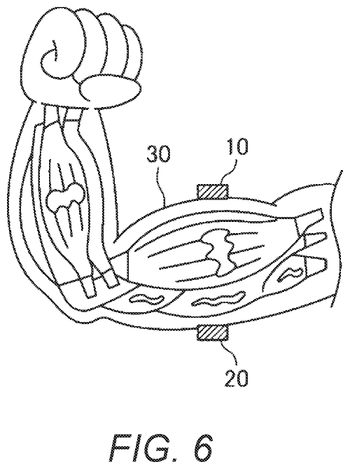

Also, in the above embodiment, when evaluating muscle fatigue at a muscle site formed of muscle fibers (brachial muscle), the two electrodes are disposed near each other along the muscle fibers, but as shown in FIG. 6, they may instead be disposed near each other so as to surround the muscle fibers.

The present invention can also be used as a muscle fatigue evaluation system. That is, the muscle fatigue evaluation system according to the present disclosure comprises at least two electrodes disposed at a predetermined interval on the living body surface, a connection means or circuit for switchably connecting a first external resistor and a second external resistor in parallel between the two electrodes, a voltage measurement means or circuit for measuring the first voltage V₁ generated when the first external resistor is connected in parallel between the two electrodes by the connecting means or circuit, and the second voltage V₂ generated when the second external resistor is connected in parallel between the two electrodes by the connecting means or circuit, and an impedance calculation means or impedance calculator for calculating the bioimpedance between the two electrodes at a muscle site under the living body surface based on the voltage ratio V₁/V₂ between the first voltage V₁ and the second voltage V₂. The local muscle fatigue at the muscle site is then evaluated based on the change over time in the calculated bioimpedance.

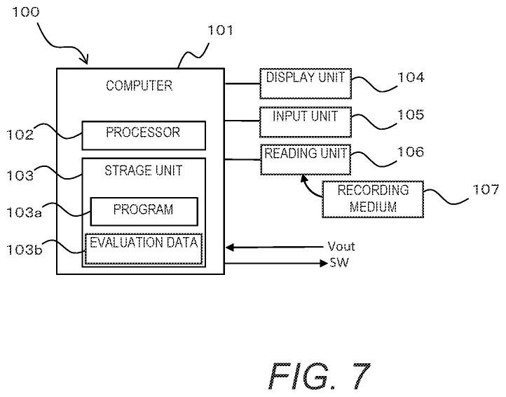

The muscle fatigue evaluation method according to the present embodiment can also be implemented by, for example, a device configuration as shown in FIG. 7. The computer 101 is configured to execute the program 103a. By causing the computer 101 to execute the program 103a, the muscle fatigue evaluation system or device 100 is configured. In particular, the computer 101 is connected to the device shown in FIG. 1, and controls the switch SW at a predetermined timing to switchably connect the first external resistor Rg1 and the second external resistor Rg2 in parallel between the two electrodes 10 and 20. The computer 101 also obtains the output voltage Vout of the amplifier 40 (i.e., the first voltage V₁ and the second voltage V₂), and calculate the bioimpedance Zb based on the formula (1) or (4). Then, the computer 101 also evaluates the muscle fatigue or local muscle fatigue based on the calculated bioimpedance according to the muscle fatigue evaluation method of the present embodiment. Thus, the computer 101 forms the impedance calculation means or impedance calculator configured to calculate the bioimpedance Zb between the two electrodes 10 and 20 at the muscle site under the living body surface 30 based on the voltage ratio V₁/V₂ between the first voltage V₁ and the second voltage V₂. The computer 101 also forms an evaluation means or evaluator configured to evaluate the muscle fatigue at the muscle site based on the change over time in the calculated bioimpedance Zb. Part or all of the processing performed by causing the computer 101 to execute the program 103a may be performed by a hardware such as a dedicated arithmetic circuit or the like.

As illustrated in FIG. 7, the computer 101 includes at least one processor 102 having a CPU (Central Processing Unit) and the like, a storage unit 103 (computer memory) having a ROM (Read Only Memory), a RAM (Random Access Memory) 103, a storage device, and the like. The storage device is, for example, a hard disk drive, a semiconductor storage device and the like.

The computer 101 can perform the muscle fatigue evaluation by causing the processor 102 to execute the program 103a stored in the storage unit 103. In addition to being read from the recording medium 107, the program 103a can be provided from an external server or the like via a transmission path such as a network (the Internet) or a LAN (Local Area Network). The recording medium 107 is an example of a non-transitory computer-readable recording medium, such as an optical disk, a magnetic disk, a nonvolatile semiconductor memory, and stores the program 103a therein.

In addition to the program 103a, various types of evaluation data 103b used for performing the muscle fatigue evaluation are stored in the storage unit 103. The evaluation data 103b stored in the storage unit 103 includes various types of thresholds used for the muscle fatigue evaluation method of the present embodiment.

Furthermore, as illustrated in FIG. 7, the computer 101 also includes a display unit 104 (display) such as a liquid crystal display device, an input unit 105 having an input device such as a keyboard and a mouse, and a reading unit 106 (reader) for reading the program 103a and various data from the recording medium 107. The reading unit 106 is a reader device or the like corresponding to the type of the recording medium 107. The thresholds used for the muscle fatigue evaluation method of the present embodiment can be input by the user using the input unit 105. The evaluation data 103b may be read out from the recording medium created by the user or may be created by the user on an external server or the like and acquired from the external server via the transmission path.

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” or “element” when used in the singular can have the dual meaning of a single part or a plurality of parts unless otherwise stated.

While only selected embodiments have been chosen to illustrate the present invention, it will be apparent to those skilled in the art from this disclosure that various changes and modifications can be made herein without departing from the scope of the invention as defined in the appended claims. For example, unless specifically stated otherwise, the size, shape, location or orientation of the various components can be changed as needed and/or desired so long as the changes do not substantially affect their intended function. Unless specifically stated otherwise, components that are shown directly connected or contacting each other can have intermediate structures disposed between them so long as the changes do not substantially affect their intended function. The functions of one element can be performed by two, and vice versa unless specifically stated otherwise. The structures and functions of one embodiment can be adopted in another embodiment. It is not necessary for all advantages to be present in a particular embodiment at the same time. Every feature which is unique from the prior art, alone or in combination with other features, also should be considered a separate description of further inventions by the applicant, including the structural and/or functional concepts embodied by such feature(s). Thus, the foregoing descriptions of the embodiments according to the present invention are provided for illustration only, and not for the purpose of limiting the invention as defined by the appended claims and their equivalents.

What is claimed is:

1. A muscle fatigue evaluation method comprising:
disposing at least two electrodes at a predetermined interval on a living body surface;
measuring a first voltage V₁ generated when a first external resistor is connected in parallel between the two electrodes and a second voltage V₂ generated when a second external resistor is connected in parallel between the two electrodes;
calculating a bioimpedance between the two electrodes at a muscle site under the living body surface based on a voltage ratio V₁/V₂ between the first voltage V₁ and the second voltage V₂; and
evaluating local muscle fatigue at the muscle site based on a change over time in the calculated bioimpedance.

2. The muscle fatigue evaluation method according to claim 1, wherein
the bioimpedance Zb is measured based on the following formula (1):
$${Z}_{b} = \frac{{R}_{g 1} \cdot {R}_{g 2}}{{R}_{g 1} - {R}_{g 2} \cdot \frac{{V}_{1}}{{V}_{2}}} ( \frac{{V}_{1}}{{V}_{2}} - 1 ) \qquad ( 1 )$$
where Rg1 represents the first external resistance and Rg2 represents the second external resistance.

3. The muscle fatigue evaluation method according to claim 1, wherein
one of the first external resistance and the second external resistance has infinite resistance.

4. The muscle fatigue evaluation method according to claim 1, wherein
the two electrodes are disposed near each other on the living body surface at a muscle site formed of muscle fibers along the muscle fibers.

5. The muscle fatigue evaluation method according to claim 1, wherein
the two electrodes are disposed near each other on the living body surface at a muscle site formed of muscle fibers so as to surround the muscle fibers.

6. The muscle fatigue evaluation method according to claim 1, wherein
the bioimpedance between the two electrodes is calculated after exercise, and
the muscle fatigue is determined to have occurred when an amount of the change over time in the bioimpedance calculated after exercise is equal to or greater than a predetermined value.

7. The muscle fatigue evaluation method according to claim 1, wherein
the bioimpedance between the two electrodes is calculated before exercise to obtain fluctuation in the calculated bioimpedance, and
the muscle fatigue is evaluated based on the change over time when the bioimpedance calculated after exercise is equal to or greater than the fluctuation.

8. The muscle fatigue evaluation method according to claim 6, wherein
the muscle fatigue at a point when an initial minimal value is reached in the change over time in the calculated bioimpedance is determined to be acute muscle fatigue, and the muscle fatigue at a point when the next minimal value is reached from the minimal value is determined to be chronic muscle fatigue.

9. The muscle fatigue evaluation method according to claim 6, wherein
the muscle fatigue at a point when a duration of a minimal value in the change over time in the calculated bioimpedance is short is determined to be acute muscle fatigue, and the muscle fatigue at a point when the duration of the minimal value is long is determined to be chronic muscle fatigue.

10. A muscle fatigue evaluation system comprising:
at least two electrodes disposed at a predetermined interval on a living body surface;
a connection circuit configured to switchably connect a first external resistor and a second external resistor in parallel between the two electrodes;
a voltage measurement circuit configured to measure a first voltage V₁ generated when the first external resistor is connected in parallel between the two electrodes by the connecting circuit, and a second voltage V₂ generated when the second external resistor is connected in parallel between the two electrodes by the connecting circuit; and
an impedance calculator configured to calculate a bioimpedance between the two electrodes at a muscle site under the living body surface based on a voltage ratio V₁/V₂ between the first voltage V₁ and the second voltage V₂,
local muscle fatigue at the muscle site being evaluated based on a change over time in the calculated bioimpedance.

11. The muscle fatigue evaluation method according to claim 2, wherein
one of the first external resistance and the second external resistance has infinite resistance.

12. The muscle fatigue evaluation method according to claim 2, wherein
the two electrodes are disposed near each other on the living body surface at a muscle site formed of muscle fibers along the muscle fibers.

13. The muscle fatigue evaluation method according to claim 3, wherein
the two electrodes are disposed near each other on the living body surface at a muscle site formed of muscle fibers along the muscle fibers.

14. The muscle fatigue evaluation method according to claim 11, wherein
the two electrodes are disposed near each other on the living body surface at a muscle site formed of muscle fibers along the muscle fibers.

15. The muscle fatigue evaluation method according to claim 2, wherein
the two electrodes are disposed near each other on the living body surface at a muscle site formed of muscle fibers so as to surround the muscle fibers.

16. The muscle fatigue evaluation method according to claim 3, wherein
the two electrodes are disposed near each other on the living body surface at a muscle site formed of muscle fibers so as to surround the muscle fibers.

17. The muscle fatigue evaluation method according to claim 11, wherein
the two electrodes are disposed near each other on the living body surface at a muscle site formed of muscle fibers so as to surround the muscle fibers.

18. The muscle fatigue evaluation method according to claim 7, wherein
the muscle fatigue at a point when an initial minimal value is reached in the change over time in the calculated bioimpedance is determined to be acute muscle fatigue, and the muscle fatigue at a point when the next minimal value is reached from the minimal value is determined to be chronic muscle fatigue.

19. The muscle fatigue evaluation method according to claim 7, wherein
the muscle fatigue at a point when a duration of a minimal value in the change over time in the calculated bioimpedance is short is determined to be acute muscle fatigue, and the muscle fatigue at a point when the duration of the minimal value is long is determined to be chronic muscle fatigue.
